(19) United States
(12) Patent Application Publication
(10) Pub. No.: US 2009/0304818 A1
(43) Pub. Date: Dec. 10, 2009

(54) METHOD OF TREATMENT

(76) Inventors: Clive N. May, North Fitzroy (AU); Rinaldo Bellomo, East Ivanhoe (AU)

Correspondence Address: MORRISON & FOERSTER LLP, 755 PAGE MILL RD, PALO ALTO, CA, 94304-1018, US

(21) Appl. No.: 12/159,196

(22) PCT Filed: Dec. 20, 2006
(86) PCT No.: PCT/AU06/01952

§ 371 Date: Nov. 24, 2008

Related U.S. Application Data
(60) Provisional application No. 60/755,266, filed on Dec. 30, 2005.

(51) Int. Cl. A61K 33/04; A61K 38/08; A61P 9/00

(52) U.S. Cl. 424/702; 514/16

(57) ABSTRACT

The present invention relates generally to a method of treatment and in particular a method of treating a subject exhibiting symptoms of kidney failure or are at risk of developing same. Even more particularly, the present invention provides a method of treating kidney failure or reducing the risk of developing kidney failure in a subject such as following or during or prior to sepsis or a related condition including severe sepsis, septic shock and the systemic inflammatory response syndrome or any state of systemic or renal vasodilatation with low blood pressure and a high cardiac output with kidney failure, such as liver disease with associated kidney failure or kidney failure after cardiopulmonary bypass in patients in whom the systemic inflammatory syndrome which follows such cardiopulmonary bypass is associated with a high cardiac output and systemic or renal vasodilatation or kidney failure in other conditions which lead to the systemic inflammatory response syndrome with systemic or renal vasodilatation such as major trauma, major surgery and similar states which can induce the aforementioned systemic inflammatory response syndrome.

### BACKGROUND OF THE INVENTION

1. Field of Invention

The present invention relates generally to a method of treatment and in particular a method of treating a subject exhibiting symptoms of kidney failure or are at risk of developing same. Even more particularly, the present invention provides a method of treating kidney failure or reducing the risk of developing kidney failure in a subject such as following or during or prior to sepsis or a related condition including severe sepsis, septic shock and the systemic inflammatory response syndrome or any state of systemic or renal vasodilatation with low blood pressure and a high cardiac output with kidney failure, such as liver disease with associated kidney failure or kidney failure after cardiopulmonary bypass in patients in whom the systemic inflammatory syndrome which follows such cardiopulmonary bypass is associated with a high cardiac output and systemic or renal vasodilatation or kidney failure in other conditions which lead to the systemic inflammatory response syndrome with systemic or renal vasodilatation such as major trauma, major surgery and similar states which can induce the aforementioned systemic inflammatory response syndrome.

2. Description of Prior Art

Reference to any prior art in this specification is not, and should not be taken as, an acknowledgment or any form of suggestion that that prior art forms part of the common general knowledge in any country.

Sepsis is the most common cause of death in intensive care units in the United States (Parillo et al. Ann Intern Med 113(3):227-242, 1990). The incidence of sepsis appears to be rising due to an increase in the use of invasive procedures, immunosuppressive therapies and emerging antibiotic and virocide resistance. Sepsis is a systemic response to infection (American College of Chest Physicians, Society of Critical Care Medicine Consensus Conference, Critical Care Medicine 20(6):864-874, 1992). In the absence of infection, it is referred to as the systemic inflammatory response syndrome. Severe sepsis is defined by the presence of likely or proven infection and at least two of the following: a temperature of greater than 38° C. or less than 36° C.; a heart rate greater than 90 beats per minute; a respiratory rate of more than 20/minute or a partial CO₂ pressure of less than 32 mm Hg; and/or an alteration in white blood cell count (such as >12,000/mm³ or <4,000/mm³) together with evidence of vital organ failure (Fitch and Gossage Postgrad Med 111(3):53-66, 2002).

Septic shock is a subset of severe sepsis and is sometimes referred to as sepsis-induced hypotension that persists despite fluid resuscitation and is associated with vital organ malfunction.

Septic shock results from or is at least exacerbated by a cytokine cascade. In general, local inflammation and substances elaborated from or associated with pathogenic organisms and viruses such as endotoxins, activated neutrophils, monocytes and tissue macrophages. This results in a cascade of pro-inflammatory cytokines and other effector molecules such as IL-1, IL-8, IL-10, TNFα, prostaglandin E₁, endogenous corticosteroids and catecholamines. This cascade leads to cellular chemotaxis, endothelial injury and activation of the coagulation cascade (Fitch and Gossage 2002 supra).

An initial and often sustained cardiovascular response occurs during the systemic inflammatory response syndrome or sepsis, which results from decreased systemic vascular resistance due to agents produced by organisms, responses to viruses, and mediators such as nitric oxide (Parker et al. Crit Care Med 100(4):483-490, 1984). A similar response characterized by vasodilation and the systemic inflammatory response syndrome is also frequently induced by other forms of severe physical injury such as cardiopulmonary bypass for cardiothoracic surgery, major trauma, other major surgery. This inflammatory response induced by other forms of body injury can be indistinguishable form that of severe sepsis and septic shock and appear mediated by the same immune system cascade of cytokines and nitric oxide induced vasodilatation (Argenziano et al. J Thorac Cardiovasc Surg; 116:973-980, 1998)). If the initial cardiovascular response is uncompensated, it has been proposed that tissue hypoperfusion results, leading to cellular dysfunction, lactic acidosis and multi-organ failure, frequently ending in death.

Traditional treatment strategies for kidney failure in the settings described above have centered around the use of volume expansion and vasodilators with the aim of restoring renal blood flow and renal function (Schrier and Wang Engl J Med; 8; 351: 159-169, 2004)

However, the continuing high incidence of death due to septic or inflammatory shock is indicative that these management practices are not optimal. Furthermore, the multiple organ failure syndrome resulting from sepsis remains a major and expensive problem in ICUs around the world for which no reliably effective treatment has yet been developed. There is a need, therefore, to develop improved intervention protocols to treat sepsis, including septic shock and systemic inflammatory response syndrome and to protect failing organs, in this case, the kidney from the adverse effects of sepsis and systemic inflammation including severe sepsis, septic shock or systemic inflammation or other conditions leading to systemic and renal vasodilatation.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

All scientific citations, patents, patent applications and manufacturer's technical specifications referred to hereinafter are incorporated herein by reference in their entirety.

The present invention is predicated in part on the surprising observation that, contrary to expectation, experimental subjects exhibiting symptoms of kidney failure, such as that induced by sepsis, systemic inflammation or other related condition leading to systemic or renal vasodilatation with low blood pressure and high cardiac output with kidney failure (including hepatorenal syndrome), require blood flow to the kidneys to be regulated through the use of agents that preferentially vasoconstrict the efferent arteriole of the glomerulus (the filtering apparatus of the kidney). This is contrary to current treatment of kidney failure in severe sepsis, which emphasizes the need to give fluids and kidney vessel vasodilators.

Hence, one aspect of the present invention contemplates a method of treating a subject exhibiting symptoms of kidney failure or who are at risk of developing kidney failure. Said method comprises administering to said subject an effective amount of preferential efferent arteriolar vasoconstricting agent for a time and under conditions sufficient to facilitate an increase in urine output and to reduce kidney failure. In this setting, preferential efferent arteriolar vasoconstriction means that the efferent arteriole constricts more than the afferent arteriole.

In one embodiment, the kidney failure results from or is exacerbated by sepsis or systemic inflammation. Reference herein to “systemic inflammation” and “sepsis” includes severe sepsis, septic shock and systemic inflammatory response syndrome after major trauma, surgery or cardiopulmonary bypass or any other condition leading to systemic or renal vasodilatation with low blood pressure and high cardiac output with kidney failure.

Accordingly, another aspect of the present invention provides a method of treating a subject with systemic inflammation, sepsis or other conditions leading to systemic or renal vasodilatation with low blood pressure and a high cardiac output with kidney failure or a risk thereof, said method comprising the administration to said subject of an effective amount of a preferential efferent arteriolar vasoconstrictor for a time and under conditions sufficient for urine output to increase.

A convenient vasoconstricting agent which has a preferential action on the efferent arteriole of the glomerulus is angiotensin II (Ang II) or a homolog, derivative, analog or functional equivalent or an agonist of Ang II-AT-1 receptor interaction. The vasoconstrictor may be given alone or in combination with, for example, a nitric oxide synthase inhibitor, an antibiotic, an anti-viral agent, an isotonic crystalloid, a colloid or a free radical scavenger (such as but not limited to vitamin E, vitamin C, selenium, an NADPH oxidase inhibitor and/or a flavonoid), a calcium antagonist such as diltiazem or other agent which causes preferential vasodilatation of the afferent arteriole of the glomerulus, an antibiotic or other anti-microbial agent (microbicide) and/or an anti-viral agent (virocide).

Hence, the present invention is further directed to the use of a preferential efferent arteriolar vasoconstrictor alone or in combination with another therapeutic agent in the generation of a therapeutic protocol to treat a subject with symptoms of kidney failure or who is at risk of developing same following, during or prior to systemic inflammation or sepsis or other conditions leading to states of systemic or renal vasodilatation characterized by low blood pressure and a high cardiac output and low blood pressure.

The present invention extends to any animal or mammal but is particularly directed to the treatment of human subjects.

### BRIEF DESCRIPTION OF THE FIGURES

FIGS. 1A through C are graphical representations of a reduction in mean arterial pressure (A); an increase in renal blood flow (B); and a reduction in urine output (C); in sepsis and the ability of Ang II infusion to return these variables to normal in four (4) conscious sheep: I, pre-sepsis control; II, sepsis control period; III, sepsis+Ang II infusion period.

### DETAILED DESCRIPTION OF THE INVENTION

Before describing the present invention in detail, it is to be understood that unless otherwise indicated, the subject invention is not limited to specific formulations of components, manufacturing methods, dosage regimes, or the like, as such may vary. It is also to be understood that the terminology used herein is for the purpose of describing particular embodiments only, and is not intended to be limiting.

In describing and claiming the present invention, the following terminology is used in accordance with the definitions set forth below.

The singular forms “a”, “an” and “the” include plural aspects unless the context clearly dictates otherwise. Thus, for example, reference to “a vasoconstrictor” includes a single vasoconstrictor, as well as two or more vasoconstrictors; reference to “an agent” includes a single agent, as well as two or more agents; reference to “the method” includes a single method or a combination of methods; and so on.

The terms “compound”, “agent”, “active agent”, “chemical agent”, “pharmacologically active agent”, “medicament”, “active” and “drug” are used interchangeably herein to refer to a chemical compound that induces a desired pharmacological and/or physiological effect that is, preferential efferent arteriole vasoconstriction causing an increase in glomerular filtration rate and restoring urine output. The terms also encompass pharmaceutically acceptable and pharmacologically active ingredients of those active agents specifically mentioned herein including but not limited to salts, esters, amides, prodrugs, active metabolites, analogs and the like. When the terms “compound”, “agent”, “active agent”, “chemical agent” “pharmacologically active agent”, “medicament”, “active” and “drug” are used, then it is to be understood that this includes the active agent per se as well as pharmaceutically acceptable, pharmacologically active salts, esters, amides, prodrugs, metabolites, analogs and the like.

Reference to a “compound”, “agent”, “active agent”, “chemical agent” “pharmacologically active agent”, “medicament”, “active”, “drug” and “pro-drug” includes combinations of two or more actives such as a vasoconstrictor and one or more of a nitric oxide synthase inhibitor, an antibiotic, an anti-viral agent, an isotonic crystalloid, a colloid or a free radical scavenger (such as but not limited to vitamin E, vitamin C, selenium, an NADPH oxidase inhibitor and/or a flavonoid), another vasosuppressor, a calcium antagonist such as diltiazem or other agent which causes preferential vasodilatation of the afferent arteriole of the glomerulus, an antibiotic or other anti-microbial agent (microbicide) and/or an anti-viral agent (virocide). A “combination” also includes multi-part compositions such as a two-part composition where the agents are provided separately and given or dispensed separately or admixed together prior to dispensation. A particular example includes angiotensin II (Ang II) and the vasosuppressor, noradrenaline.

For example, a multi-part pharmaceutical pack may have two or more active agents separately maintained. The pack may also be designed to facilitate administration of the active ingredients.

The terms “effective amount” and “therapeutically effective amount” of an agent as used herein mean a sufficient amount of the agent (e.g. a vasoconstrictor) to provide the desired therapeutic or physiological effect or outcome. The desired outcome is a reduction in kidney failure as measured by an increase in urine output and an improved creatinine clearance to normal or premorbid levels. Undesirable effects, e.g. side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation.

By “pharmaceutically acceptable” carrier, excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Similarly, a “pharmacologically acceptable” salt, ester, emide, prodrug or derivative of a compound as provided herein is a salt, ester, amide, prodrug or derivative that this not biologically or otherwise undesirable.

The terms “treating” and “treatment” as used herein refer to reduction in severity and/or frequency of symptoms of the condition being treated, elimination of symptoms and/or underlying cause, prevention of the occurrence of symptoms of the condition and/or their underlying cause and improvement or remediation or amelioration of damage following a condition.

“Treating” a subject may involve prevention of a condition or other adverse physiological event in a susceptible individual as well as treatment of a clinically symptomatic individual by ameliorating the symptoms of the condition. Conveniently, treatment may be initiated immediately a subject's urine output drops or an infection or other condition is diagnosed. It is proposed that treatment commence after diagnosis of sepsis or other related conditions leading to a state of systemic vasodilatation with low blood pressure and high cardiac output with evidence of kidney failure or risk thereof. Hence, the present invention encompasses treating a subject having a risk of developing kidney failure.

A “subject” as used herein refers to an animal, preferably a mammal and more preferably human who can benefit from the pharmaceutical formulations and methods of the present invention. There is no limitation on the type of animal that could benefit from the presently described pharmaceutical formulations and methods. A subject regardless of whether a human or non-human animal may be referred to as an individual, subject, patient, animal, host or recipient. The compounds and methods of the present invention have particular application in human medicine but also in veterinary medicine as well as in general, domestic or wild animal husbandry.

As indicated above, the preferred animals are humans or other primates such as orangutans, gorillas, marmosets, livestock animals, laboratory test animals, companion animals or captive wild animals, as well as avian species.

Examples of laboratory test animals include mice, rats, rabbits, sheep, simian animals, guinea pigs and hamsters. Rabbits, rodent, sheep and simian animals provide a convenient test system or animal model. Livestock animals include sheep, cows, pigs, goats, horses and donkeys.

Hence, one aspect of the present invention contemplates a method of treating a subject exhibiting symptoms of kidney failure or who are at risk of developing kidney failure, said method comprises administering to said subject an effective amount of preferential efferent arteriolar vasoconstricting agent for a time and under conditions sufficient to facilitate an increase in urine output and to reduce kidney failure.

In this context, preferential efferent arteriolar vasoconstriction means that the efferent arteriole constricts more than the afferent arteriole.

In one embodiment, the kidney failure results from or is exacerbated by systemic inflammation or sepsis. Reference herein to systemic inflammation or “sepsis” includes severe sepsis, septic shock and the systemic inflammatory response syndrome or any other condition leading to systemic or renal vasodilatation with low blood pressure and high cardiac output with kidney failure.

A preferential efferent arteriolar vasoconstrictor includes any agent which reduces blood flow, particularly to the kidneys. An example of a particularly useful vasoconstrictor that has a preferential action on the efferent arteriole compared to the afferent arteriole is angiotensin II (Ang II) or a homolog, derivative, analog or functional equivalent thereof or an agonist of an Ang II interaction with its receptor, the AT-1 receptor (angiotensin AT-1 receptor agonist) or a combination of Ang II or Ang II AT-1 receptor agonist and another vasosuppressor such as noradrenaline.

Reference to “Ang II” or “angiotensin AT-1 receptor agonist” or “Ang II AT-1 receptor agonist” includes derivatives or homologs which have a longer half-life such as a longer serum or tissue fluid half-life.

Hence, the present invention contemplates a method of treating a subject exhibiting symptoms of kidney failure or who are at risk of developing kidney failure, said method comprising administering to said subject an effective amount of Ang II or a homolog, derivative, analog or functional equivalent or an agonist of Ang II-AT-1 receptor interaction for a time and under conditions sufficient to facilitate an increase in urine output and reduce kidney failure.

In another embodiment, the present invention provides a method of treating a subject with systemic inflammation or sepsis or other conditions leading to systemic or renal vasodilatation with low blood pressure and a high cardiac output with kidney failure or a risk thereof, said method comprising administering to said subject an effective amount of a preferential efferent arteriolar vasoconstrictor for a time and under conditions for urine output to increase and kidney failure to resolve.

As indicated above, the subject may be any animal or mammal but is preferably a human. The present invention further extends to unborn foetuses such as when a pregnant subject is diagnosed with sepsis or related conditions leading to a state of systemic and renal vasodilation.

Hence, in a preferred embodiment, the present invention provides a method of treating a subject with systemic inflammation, sepsis, septic shock or other related conditions leading to systemic and renal vasodilatation with low blood pressure and high cardiac output and a failing kidney or risk thereof, said methods comprising administering to said subject an effective amount of Ang II or a homolog, derivative, analog or functional equivalent or an agonist of Ang II-AT-1 receptor interaction for a time and under conditions for urine output to increase and reduce kidney failure.

A further embodiment comprises the administration of Ang II and/or an Ang II AT-1 receptor agonist and another vasosuppressor such as noradrenaline.

The term “severe sepsis” refers to an acute bacterial, microbial or viral infection leading to a cascade of cytokines and inflammatory mediators being activated including the complement system, the coagulation cascade, the fibrinolytic system, catecholamines, glucocorticoids, prekallikrein, bradykinin, histamines, β-endorphins, encephalins, adrenocorticoid hormone, circulating myocardial depressant factors, cachectin (tumor necrosis factor), interleukins, other cytokines and nitric oxide. Symptoms of severe shock include fever, chills, constitution malaise, anxiety and/or confusion. Generally, the subject will exhibit a temperature of greater than 38° C. or less than 36° C.; a heart rate greater than 90 beats per minute; a respiratory rate of more than 20/minute or a partial CO₂ pressure of less than 32 mm Hg; and/or an alteration in white blood cell count (such as >12,000/mm³ or <4,000/mm³). For severe sepsis to exist, the patient must also have evidence of vital organ failure (low urine output, high blood lactate levels, delirium, lung malfunction, low platelet count etc.). If the blood pressure is very low and does not improve with fluids the condition is called septic shock.

The active agents may be administered for therapy by any suitable route. Suitable routes of administration may include oral, rectal, nasal, inhalation of aerosols or particulates, topical (including buccal and sublingual), transdermal, vaginal, intravesical and parenteral (including subcutaneous, intramuscular, intravenous, intrasternal, intrathecal, epidural and intradermal). It will be appreciated that the preferred route will vary with the condition and age of the subject, the nature of the condition being treated, its location within the subject and the judgement of the physician or veterinarian. It will also be understood that individual active agents may be administered by the same or different distinct routes.

As discussed above, an “effective amount” refers to an amount of active agent that provides the desired therapeutic or physiological outcome such as reducing kidney failure as endured by returning urine output to normal levels. Dosing may occur at intervals of several seconds, minutes, hours, days, weeks or months. Suitable dosage amounts and regimes can be determined by the attending physician or veterinarian. For example, Ang II or an angiotensin AT-1 receptor agonist or pharmaceutically acceptable salts, derivatives, homologs, analogs or functional equivalents thereof, may be administered generally by infusion to a subject at a rate of between about 0.01 μg/kg/min to about 20 mg/kg/min continuously for from about 1 hour (or less) to up to about 500 or more hours, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499 or 500 hours. Dosing of the efferent arteriolar vasoconstrictor, such as an Ang II, can be determined by the attending physician in accordance with dosing rates in practice.

The present invention further contemplates combination therapy. Hence, a preferential efferent arteriolar vasoconstrictor may be given with a nitric oxide synthase inhibitor, an antibiotic, an anti-viral agent, an isotonic crystalloid, a colloid or a free radical scavenger (such as but not limited to vitamin E, vitamin C, selenium, an NADPH oxidase inhibitor and/or a flavonoid), another vasosuppressor, a calcium antagonist such as diltiazem or other agent which causes preferential vasodilatation of the afferent arteriole of the glomerulus, an antibiotic or other anti-microbial agent (microbicide) and/or an anti-viral agent (virocide).

The term “antibiotic” is used in its broadest sense and includes cell-produced molecules as well as chemotherapeutic agents. A convenient reference is MIMS Annual, CMPMedica Australia Pty Limited:1-1700, 2005, Publisher C & C Offset Printing Co., Ltd, Hong Kong.

In combination therapy, the two or more active agents may be administered simultaneously or sequentially. If simultaneously, the agents may be in the same or different formulations. If sequentially, they may be administered with nanoseconds, seconds, minutes, hours or days of each other.

Hence, another aspect of the present invention contemplates a method of treating a subject exhibiting symptoms of kidney failure or at risk of developing same, said method comprising administering to said subject an effective amount of a preferential efferent arteriolar vasoconstricting agent and at least one other agent selected from a nitric oxide synthase inhibitor, an antibiotic, an anti-viral agent, an isotonic crystalloid, a colloid or a free radical scavenger (such as but not limited to vitamin E, vitamin C, selenium, an NADPH oxidase inhibitor and/or a flavonoid), another vasosuppressor, a calcium antagonist such as diltiazem or other agent which causes preferential vasodilatation of the afferent arteriole of the glomerulus, an antibiotic or other anti-microbial agent (microbicide) and/or an anti-viral agent (virocide) for a time and under conditions sufficient to facilitate an increase in urine output and to reduce kidney failure. An example of another vasosuppressor is noradrenaline. Examples of a preferential efferent arteriolar vasoconstricting agent is Ang II or an Ang II AT-1 receptor agonist.

In a related embodiment, the present invention provides a method of treating a subject with systemic inflammation, sepsis including severe sepsis and septic shock, said method comprising administering to said subject an effective amount of a preferential efferent arteriolar vasoconstrictor and at least one other agent selected from a nitric oxide synthase inhibitor, an antibiotic, an anti-viral agent, an isotonic crystalloid, a colloid or a free radical scavenger (such as but not limited to vitamin E, vitamin C, selenium, an NADPH oxidase inhibitor and/or a flavonoid), a calcium antagonist such as diltiazem or other agent which causes preferential vasodilatation of the afferent arteriole of the glomerulus, an antibiotic or other anti-microbial agent (microbicide) and/or an anti-viral agent (virocide) flavonoid for a time and under conditions for urine output to increase.

Accordingly, still another aspect of the present invention contemplates a treatment protocol for systemic inflammation, sepsis including the systemic inflammatory syndrome, severe sepsis, septic shock and any condition characterized by a state of systemic or renal dilatation with low blood pressure and high cardiac output and kidney failure or a risk thereof in a subject, said protocol comprising the steps of identifying renal failure or a risk thereof, administering to said subject an effective amount of a preferential efferent arteriolar vasoconstrictor and a nitric oxide synthase inhibitor, an antibiotic, an anti-viral agent, an isotonic crystalloid, another vasosuppressor, a colloid or a free radical scavenger (such as but not limited to vitamin E, vitamin C, selenium, an NADPH oxidase inhibitor and/or a flavonoid), a calcium antagonist such as diltiazem or other agent which causes preferential vasodilatation of the afferent arteriole of the glomerulus, an antibiotic or other anti-microbial agent (microbicide) and/or an anti-viral agent (virocide) effective to reduce the level or risk of renal failure.

In a particular embodiment, the vasoconstrictor is Ang II and/or Ang II AT-1 receptor agonist and the other vasosuppressor is noradrenaline.

The present invention also relates to compositions comprising a preferential efferent arteriolar vasoconstrictor or a pharmaceutically acceptable salt, derivative, homolog or analog thereof, optionally with another agent such as a nitric oxide synthase inhibitor, an antibiotic, an anti-viral agent, an isotonic crystalloid, another vasosuppressor, a colloid or a free radical scavenger (such as but not limited to vitamin E, vitamin C, selenium, an NADPH oxidase inhibitor and/or a flavonoid), a calcium antagonist such as diltiazem or other agent which causes preferential vasodilatation of the afferent arteriole of the glomerulus, an antibiotic or other anti-microbial agent (microbicide) and/or an anti-viral agent (virocide) together with one or more pharmaceutically acceptable additives and optionally other medicaments.

The pharmaceutically acceptable additives may be in the form of carriers, diluents, adjuvants and/or excipients and they include all conventional solvents, dispersion agents, fillers, solid carriers, coating agents, antifungal or antibacterial agents, dermal penetration agents, surfactants, isotonic and absorption agents and slow or controlled release matrices. The active agents may be presented in the form of a kit of components adapted for allowing concurrent, separate or sequential administration of the active agents. Each carrier, diluent, adjuvant and/or excipient must be “pharmaceutically acceptable” in the sense of being compatible with the other ingredients of the composition and physiologically tolerated by the subject. The compositions may conveniently be presented in unit dosage form and may be prepared by methods well known in the art of pharmacy. Such methods include the step of bringing into association the active ingredient with the carrier, which constitutes one or more accessory ingredients. In general, the compositions are prepared by uniformly and intimately bringing into association the active ingredient with liquid carriers, diluents, adjuvants and/or excipients or finely divided solid carriers or both, and then if necessary shaping the product.

Generally, the compositions of the present invention are suitable for intravenous administration such as a continual or intermittent infusion.

The present invention does extend, however, to other forms of compositions such as for parenteral, oral, intraperitoneal, subcutaneous, intramuscular, nasal, intrathecal or intraocular administration.

The compounds of the subject invention may also be administered in a sustained (i.e. controlled) or slow release form. A sustained release preparation is one in which the active ingredient is slowly released within the body of the subject once administered and maintains the desired drug concentration over a minimum period of time. The preparation of sustained release formulations is well understood by persons skilled in the art. Dosage forms may include oral forms, implants and transdermal forms. For slow release administration, the active ingredients may be suspended as slow release particles or within liposomes, for example.

The pharmaceutical compositions of the present invention may be packaged for sale with other active agents or alternatively, other active agents may be formulated with Ang II or Ang II AT-1 receptor agonistor its pharmaceutical salts, derivatives, homologs or analogs thereof alone or in combination with one or more of a nitric oxide synthase inhibitor, an antibiotic, an anti-viral agent, an isotonic crystalloid, another vasosuppressor, a colloid or a free radical scavenger (such as but not limited to vitamin E, vitamin C, selenium, an NADPH oxidase inhibitor and/or a flavonoid), a calcium antagonist such as diltiazem or other agent which causes preferential vasodilatation of the afferent arteriole of the glomerulus, an antibiotic or other anti-microbial agent (microbicide) and/or an anti-viral agent (virocide).

The present invention creates a tamper-proof vasoconstricting delivery system that provides for full delivery of a preferential efferent arteriolar vasoconstrictor (such as Ang II or an Ang II AT-1 receptor agonist) and an agent selected from a nitric oxide synthase inhibitor, an antibiotic, an anti-viral agent, an isotonic crystalloid, another vasosuppressor, a colloid or a free radical scavenger (such as but not limited to vitamin E, vitamin C, selenium, an NADPH oxidase inhibitor and/or a flavonoid), a calcium antagonist such as diltiazem or other agent which causes preferential vasodilatation of the afferent arteriole of the glomerulus, an antibiotic or other anti-microbial agent (microbicide) and/or an anti-viral agent (virocide) while at the same time effectively eliminating the problem of tampering by diversion, adulteration, or pulverization of the medication for abuse by addicts.

The formulation may also contain carriers, diluents and excipients. Details of pharmaceutically acceptable carriers, diluents and excipients and methods of preparing pharmaceutical compositions and formulations are provided in Remmingtons Pharmaceutical Sciences 18^{th} Edition, 1990, Mack Publishing Co., Easton, Pa., USA.

The active agents for use in the present invention may also be presented for use in veterinary compositions. These may be prepared by any suitable means known in the art. Examples of such compositions include those adapted for:
(a) oral administration, e.g. drenches including aqueous and non-aqueous solutions or suspensions, tablets, boluses, powders, granules, pellets for admixture with feedstuffs, pastes for application to the tongue;
(b) parenteral administration, e.g. subcutaneous, intramuscular or intravenous injection as a sterile solution or suspension or through intra-nasal administration;
(c) topical application, e.g. creams, ointments, gels, lotions, etc.

Preferably, however, the composition is adopted for intravenous infusion.

The present invention will now be further described with reference to the following examples, which are intended for the purpose of illustration only and are not intended to limit the generality of the subject invention as hereinbefore described.

### Example 1

### Animal Model

Seven female Merino ewes weighing between 34.2 kg and 47.3 kg were procured for chronic instrumentation. The sheep were held and studied in metabolic cages, with free access to food and water. The animals underwent two separate operative procedures. For all procedures, anaesthesia was induced with sodium thiopentone (15 mg/kg) for endotracheal tube placement (cuff size 10). Maintenance anaesthesia was by means of oxygen/air/isoflurane (1 to 2%). Fractional inspired oxygen was altered to maintain PaO₂ at approximately 100 mm Hg, and ventilation was controlled to maintain PaCO₂ at approximately 40 mm Hg.

First, a left-sided thoracotomy was performed. The pericardium was opened, and a transit time flow probe (20 mm, Transonics Systems, Ithaca, N.Y.) was placed around the pulmonary artery to measure cardiac output. After two weeks recovery, a left-sided flank incision was made and a retroperitoneal dissection was performed to expose the left renal artery. A transit time flow probe (4 mm, Transonics Systems, Ithaca, N.Y.) was placed around the renal artery. During the same operative procedure, a single carotid artery loop was created. The animals were allowed to recover for two weeks before experimentation.

The transit-time flow probes were connected to flow meters (Transonics Systems, Ithaca, N.Y.). Before starting the experiment, a Tygon catheter (Cole-Parmers; Boronia, Australia, id 1.0 mm, od 1.7 mm) was inserted into carotid loop to measure arterial pressure. Two internal jugular venous polyethylene catheters (Critchley, Silverwater, Australia, id 1.2 mm, od 1.7 mm) were placed to measure central venous pressure and for infusion. The cannulae were connected to pressure transducers (TDXIII, Code, Lakewood, Colo.) tied to the wool on the back. A correction factor was added in the data acquisition program to correct for the height of the transducers above the heart (Labview National Instruments). A urinary catheter was inserted for measurement of urine volume and for sample collection.

Analog signals of mean arterial pressure (MAP), central venous pressure (CVP), cardiac output (CO), and renal blood flow (RBF) were collected using a PC using a customized data acquisition system (Labview National Instruments). Data were recorded at 100 Hz for 10 s at every minute throughout the experimental protocol. Total peripheral conductance (TPC) (cardiac output/mean arterial pressure) and renal vascular conductance (RVC) (renal blood flow/mean arterial pressure) were calculated.

### Example 2

### Protocol and Measurements

During the experimental periods MAP, CVP, CO, RBF and heart rate were measured continuously. Following a 12-hour baseline period, sheep were monitored for a 48 hour control period. After 7 days, sheep were monitored for a 2^{nd} baseline period, followed by induction of sepsis by intravenous administration of a bolus of live E. coli (3.9×10⁹ colony forming units in 15 mL saline), followed by a continuous infusion (1.7×10¹ colony forming units at 5 mL/h) for 48 h. The E. coli bacteria, had been originally isolated from blood cultures from a septic patient, were grown from a stock kept at −70° C. and incubated overnight in broth. The culture medium was then adjusted by turbidometry to give the correct concentration of bacteria.

In both treatments, after the 12-hour baseline period, normal saline (1 mL/kg/h) was infused to avoid hypovolemia. Urinary output was measured and urine sampled every 90 minutes. Arterial blood samples for analysis of creatinine and urine samples to measure creatinine were measured 12-hourly. No fluid boluses, inotropic support, mechanical ventilation, or antibiotics were administered. The animals were conscious and not sedated for the duration of the experiment. At the end of the septic period, the sheep were sacrificed using the intravenous administration of sodium pentobarbitone (150 mg/kg).

### Example 3

### Statistical Analysis

Data are presented as mean±standard deviation. In the control and the sepsis group mean values for each hour were compared using non-parametric 2-way repeated measures ANOVA (Friedman's test). The control and septic group values for central and renal hemodynamics and renal functional variables were compared using ANOVA with the Bonferroni post-hoc test A p<0.05 was considered statistically significant.

### Example 4

### Assessment of Renal Blood Flow and Renal Vascular Conductance in Experimental Septic Acute Renal Failure (ARF)

In six of the seven sheep, the E. coli infusion was continued for 48 h. The sheep developed tachypnea, tachycardia and a temperature of >41° C., and began to use the accessory muscles of respiration. The white blood cells decreased after 48 h of E. coli infusion to 1,600±800/μL compared with 5400±2900/μL in the control group (P<0.05). One sheep died 12 h after the induction of sepsis.

### Systemic Hemodynamic Pattern

Administration of E. coli induced hyperdynamic sepsis with a delayed onset. After 10 h of E. coli infusion, CO had increased significantly and continued to increase throughout the infusion to a maximum of 9.8±1.1 L/min, compared with 3.8±0.4 L/min with the control treatment (p<0.05). Significant hypotension occurred at the same time and blood pressure continued to decrease during the infusion of E. coli (89.2±3.2 mm Hg vs 64.3±5.3 mm Hg; P<0.05). Heart rate increased rapidly in response to administration of E. coli reaching a plateau at 9 hours that was maintained for the 48 hour infusion (65.0±7.3 vs 161.1±18.3 beats/min; p<0.05). There was marked peripheral vasodilatation as shown by the increase in total peripheral conductance (TPC), which reached significance 10 hours after E. coli injection and reached a maximum of 153.7±24.7 mL/min/mmHg, compared with 42.8±3.5 mL/min/mmHg during the control treatment (p<0.05). The CVP tended to increase in both groups without reaching a significant difference.

### Renal Hemodynamic and Functional Parameters

At 3 hours after the injection of E. coli, RBF increased transiently over the following 6 hours, returning to baseline by 12 hours. RBF then began to increase again after 19 h, reaching a maximum of 757.4±250.1 mL/min after 45 h compared to control value of 262.3±47.7 mL/min (p<0.05). This change was dependent on increased renal vascular conductance (3.0±0.7 mL/min/mmHg vs 11.4 mL/min/mmHg; p<0.05.

After 24 hours, the serum creatinine significantly increased in the sepsis group, reaching a value of 325±153 μmol/L compared with 73±18 μmol/L during the control treatment. The serum creatinine in the control group was unaltered during the experiment. The glomerular filtration rate (GFR) decreased after E. coli infusion (20.1±19.3 mL/min) compared to the control group (95.5±25.9 mL/min) (p<0.05). Urinary output, increased briefly after the induction of sepsis and then decreased after 21 hours to below 0.5 mL/h/kg. During the 24-48 hour period hourly urinary output was 1.4 ml/h/kg in controls compared to 0.3 ml/h/kg.

### Example 5

### Effects of Angiotensin II (Ang II)

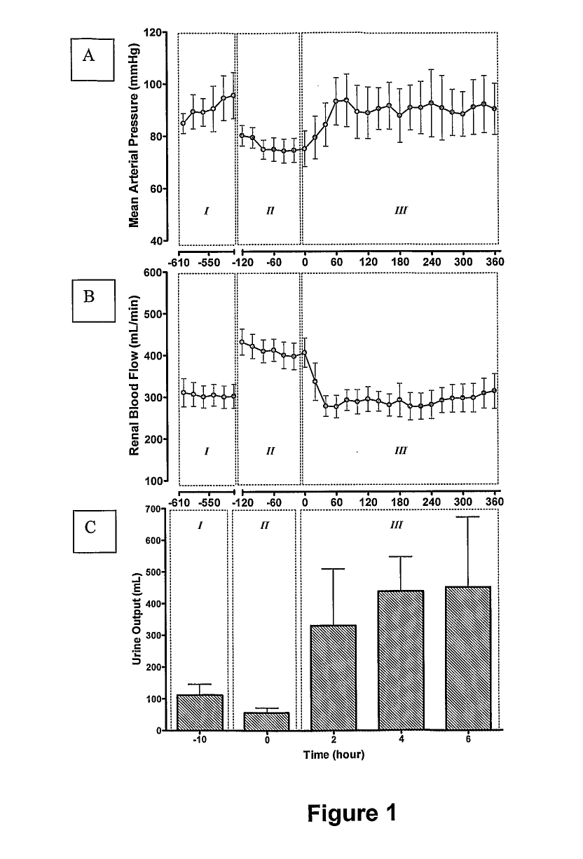

FIG. 1 shows the effects of administering Ang II to a mammal. The data show a reduction in mean arterial pressure, an increase in renal blood flow and a reduction in urine output in sepsis and the ability of Ang II infusion to return these variables to normal in four (4) conscious sheep.

### Example 6

### Effects of Ang II in Short Term Sepsis

The experiment in Example 4 is repeated in a short-term sepsis experiment with likely similar results.

### Example 7

### Effects of Ang II in Long Term Sepsis

The experiment in Example 5 is repeated in a long-term sepsis experiment with likely similar results.

### Example 8

### Combination Therapy

The experiments are repeated with a nitric oxide synthase inhibitor.

### Example 9

### Determining Renal ATP Levels

Using the experimental methods described in Examples 1 through 8, magnetic resonance spectroscopy is used to determine changes in renal ATP in sepsis and following treatment with Ang II.

### Example 10

### Clinical Studies

Clinical trials are conducted using patients diagnosed with or suspected of having or at high risk of developing systemic inflammation, sepsis or a related condition. Renal blood flow is measured as are urine output and mean arterial pressure. Results similar to those shown in FIG. 1 are expected.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

American College of Chest Physicians, Society of Critical Care Medicine Consensus Conference, Critical Care Medicine 20(6):864-874, 1992
Argenziano M, Chen J M et al J Thorac Cardiovasc Surg 1998 116: 973-980, 1998
Fitch and Gossage Postgrad Med 111(3):53-66, 2002
MIMS Annual, CMPMedica Australia Pty Limited:1-1700, 2005, Publisher C & C Offset Printing Co., Ltd, Hong Kong
Parillo et al. Ann Intern Med 113(3):227-242, 1990
Parker et al. Crit Care Med 100(4):483-490, 1984
Remmingtons Pharmaceutical Sciences 18^{th} Edition, 1990, Mack Publishing Co., Easton, Pa., USA.
Schrier R W, and Wang W. N Engl J Med 2004; 8; 351: 159-169, 2004

1. A method of treating a subject exhibiting symptoms of acute kidney failure or who is at risk of developing acute kidney failure, said method comprising administering to said subject an effective amount of a preferential efferent arteriolar vasoconstricting agent for a time and under conditions sufficient to facilitate an increase in urine output and to reduce kidney failure.

2. The method of claim 1 wherein the acute kidney failure is associated with systemic inflammation, sepsis or other related conditions leading to systemic and renal vasodilatation with low blood pressure and high cardiac output.

3. The method of claim 2 wherein the sepsis is septic shock or severe sepsis.

4. The method of claim 2 wherein the sepsis is systemic inflammatory response syndrome.

5. The method of claim 2 wherein the systemic inflammatory response syndrome is a condition capable of causing systemic inflammation and systemic or renal vasodilatation including but not confined to the systemic inflammatory syndrome after cardiopulmonary bypass, major trauma, major surgery or related conditions.

6. The method of claim 1 wherein the subject is human.

7. The method of claim 1 wherein the preferential efferent arteriolar vasoconstricting agent is angiotensin II (Ang II) or a pharmaceutically acceptable salt, derivative, homolog, analog or functional equivalent thereof.

8. The method of claim 1 wherein the preferential efferent arteriolar vasoconstricting agent is an agonist of Ang II interaction with its receptor, AT-1.

9. The method of claim 1 further comprising the administration of an agent selected from a nitric oxide synthase inhibitor, an antibiotic, an anti-viral agent, an isotonic crystalloid, another vasosuppressor, a colloid or a free radical scavenger, a calcium antagonist or other agent which causes preferential vasodilatation of the afferent arteriole of the glomerulus, an antibiotic or other anti-microbial agent (microbicide) and/or an anti-viral agent (virocide).

10. The method of claim 9 wherein the other vasosuppressor is noradrenaline.

11-25. (canceled)

26. The method of claim 9 wherein the free radical scavenger is vitamin E, vitamin C, selenium, or NADPH oxidase inhibitor or a flavenoid.

27. The method of claim 9 wherein the calcium antagonist is diltiazem.
